(19) United States
(12) Patent Application Publication
(10) Pub. No.: US 2021/0307675 A1
(43) Pub. Date: Oct. 7, 2021

(54) MOBILE DIAGNOSTIC SYSTEM FOR THE EARLY DETECTION OF VASCULAR DISEASES

(71) Applicant: Bluelife GmbH, Planegg (DE)

(72) Inventors: Martin Kriegmair, Planegg (DE); Ralph Oberneder, Planegg (DE); Irmgard Huber, Planegg (DE)

(21) Appl. No.: 17/263,709

(22) PCT Filed: Jul. 24, 2019
(86) PCT No.: PCT/EP2019/069876

§ 371 Date: Jan. 27, 2021

(30) Foreign Application Priority Data
Jul. 30, 2018 (DE) 10 2018 118 394.1

(51) Int. Cl. A61B 5/00

(52) U.S. Cl. CPC A61B 5/4393; A61B 5/7275; A61B 5/6804; A61B 2562/0261; A61B 2562/029; A61B 2562/0271; A61B 2562/0247

(57) ABSTRACT

A mobile diagnostic system (1) for the early detection of vascular diseases includes a measuring module (2) and an evaluation module (7), wherein the measuring module (2) comprises a measuring cuff (3) made of a textile material and at least one sensor (4) for measuring expansion, pressure, temperature and/or moisture, and wherein the evaluation module (7) comprises an evaluation and diagnostic unit designed to derive a biomarker identifying a vascular disease from the measured variable detected by the at least one sensor (4).

The present invention relates to a mobile diagnostic system for the early detection of vascular diseases.

In recent years, medical and pharmaceutical industries have shown growing interest in researching biomarkers, particularly for the early detection of vascular diseases. This is particularly due to vascular diseases, particularly an imminent heart attack or stroke, still to date, despite highly intensive research efforts, not being diagnosed adequately—and/or (too) late—or not even being able to be treated at all. Moreover, vascular diseases are often of a complex nature and only able to be determined very laboriously and/or arduously with conventional methods.

Given this problem, the present invention is based on the task of specifying a diagnostic system which enables early detection of vascular diseases, whereby the diagnostic system is of mobile design and in particular can be used at home.

The invention solves this task with a mobile diagnostic system according to independent claim 1, whereby advantageous developments of the inventive diagnostic system are specified in the corresponding dependent claims.

Accordingly, the present invention relates in particular to a mobile diagnostic system for the early detection of vascular diseases, wherein the diagnostic system comprises a measuring module as well as an evaluation module. The measuring module has a measuring cuff made of a textile material as well as at least one sensor, wherein the sensor is designed to measure expansion, pressure, temperature and/or moisture.

The measuring cuff serves in particular as a monitoring device for the nocturnal tumescence and stiffening of the penis. The monitoring of nocturnal penile tumescence is used as a diagnostic procedure to determine whether a given case of male impotence is psychogenic or organic in nature. The at least one sensor of the measuring cuff is therefore designed to directly or indirectly detect the occurrence of particularly a nocturnal erection of the user. The evaluation and diagnostic unit of the diagnostic system is designed to derive the probability or risk of erectile dysfunction or a biomarker identifying erectile dysfunction on the basis of the measured variables detected by the at least one sensor.

Erectile dysfunction is a serious condition. Thanks to modern testing methods, we know today that organic ailments play a role in the majority of cases. Yet when nocturnal erections occur contemporaneously, psychological causes are to be presumed.

The present invention is in particular based on the knowledge that erectile dysfunction is frequently also the harbinger of other even more serious diseases. Scientific studies show that erectile dysfunction is often an indicator of imminent heart attack and stroke, as the blood vessels of the penis are similar to those of the heart.

Many sufferers do not initially go to an andrologist or urologist out of a false sense of shame. Yet swift examination and diagnosis is often necessary to avoid long-term damage.

Today, the diagnosis of erectile dysfunction primarily lies in the hands of urologists. A neurologist or psychotherapist will be consulted if necessary. Many cases require interdisciplinary collaboration. The urologist will first clarify the exact nature of the sexual problems and how long they have existed during the course of an anamnesis interview. This provides the first signs of whether there are psychological factors leading to loss of potency. A history of risk and medication follows in order to ascertain whether the patient has had previous illnesses which might lead to erectile dysfunction or whether he is taking medication having such an effect.

A physical examination and ultrasound can provide indication of injuries; hormonal disorders can be concluded from a blood sample. If the findings are not serious, the patient is given PDE-5 inhibitors. If the treatment doesn't work or there are signs of organic damage (e.g. to vessels), more invasive methods are selected to find the cause. Included among these are:
Nocturnal Penile Tumescence and Rigidity Measurement (NPTR measurement)
Corpus Cavernosum Electromyogram (CC-EMG)
Penile Sympathetic Skin Response (PSH)
Intracavernous injection test (SKIT)
Pharmacological Phalloarteriography (PPAG)
Pharmaco-cavernosography and cavernosometry (PKMG)

The NPTR measurement records nocturnal erections, A healthy person experiences this three to six times per night for at least ten minutes. If they occur in the patient, an organic disorder is excluded, there must be psychological reasons for the erectile dysfunction. In the case of SKIT, which is also used as intracavernous injection therapy, an erection is induced with drugs (papaverine, phentolamine and prostaglandin). If it lasts for more than 15 minutes, dysfunction of the vessels can in all likelihood be ruled out. Duplex sonography performed during the blood surge provides objective data on the flow of blood as it occurs. CC-EMG and PSH measure nerve or muscle activity respectively. They thus allow the excluding of nervous and muscular disorders. If the SKIT returns a suspicious result, PPAG can be used to seek arterial causes and PKMG to seek venous causes. In PPAG, the arteries of the erectile tissue are visualized using a contrast medium and an X-ray machine. In PKMG, pressure in the penis is measured while it is being kept rigid with drugs or a saline infusion respectively. If this is only successful with a high flow of saline or not successful at all, a venous outflow disorder is surmised.

These methods have the disadvantage of generally not being able to be used on an outpatient basis or even in the patient's familiar surroundings. Furthermore, the need for medical professionals to attend to the patient is unavoidable with the methods as used to date for diagnosing erectile dysfunction.

This often leads to a sufferer shying away from testing for erectile dysfunction. If and when the diagnosis is then made, however, it is often too late for the diagnostic result to be construed as an effective biomarker for vascular disease, particularly an imminent heart attack or stroke.

With the mobile diagnostic system according to the invention, it is readily and easily possible to diagnose erectile dysfunction, or respectively detect the indicators which are indicative of erectile dysfunction, such as in particular the absence of nocturnal erections. The diagnostic system allows a sufferer to be able to perform all the steps necessary for the diagnosis in his own familiar surroundings without the involvement of medical professionals, whereby his normal living conditions are concurrently not or not substantially disrupted.

To that end, it is provided for the sensor to be designed as a measuring cuff which is slipped over the penis. The cuff consists of a textile material which can comprise stretchable fibers so that optimum wearing comfort is ensured. The at least one sensor of the diagnostic system is thereby integrated into the textile material of the measuring cuff or is even part of the textile material so as to be haptically unobtrusive.

The mobile diagnostic system further comprises an evaluation module (realized separately from the measuring module) having an evaluation and diagnostic unit. The evaluation and diagnostic unit is designed to evaluate the measured variable detected by the at least one sensor and correspondingly derive a biomarker identifying a vascular disease.

The at least one sensor of the diagnostic system can be designed as a sensor module able to be removed from the measuring cuff. However, with respect to wearing comfort, it is advantageous for the at least one sensor to be integrated into the textile material of the measuring cuff. Alternatively or additionally, it is thereto also conceivable for the sensor itself to be at least in part designed from a textile material or a similarly flexible and haptically comfortable material.

According to embodiments of the present invention, the diagnostic system comprises a memory device allocated to the measuring module in order to be able to buffer measured variables detected by the at least one sensor within a predefined or predefinable period of time. In this context, it is for example conceivable for the at least one sensor to be in or be able to be brought into a communication link with the memory device via a data line so as to transmit the measured variables detected by the at least one sensor to the memory device continuously or as needed. Having the data line either be integrated into the textile material of the measuring cuff or consisting of a corresponding material, in particular textile material, in order to convey a haptically comfortable or unobtrusive impression thereby lends itself to wearing comfort.

According to embodiments of the present invention, the memory device, which is allocated to the measuring module of the diagnostic system, comprises a suitable interface via which the measured variables of the at least one sensor buffered in the memory device can be transmitted to the evaluation and diagnostic unit of the evaluation module as needed or continuously, preferably wirelessly.

This separating of the measuring module and the evaluation module particularly enables the fewest possible components to touch or respectively be placed on the user's body in order to improve wearing comfort.

So that the measuring cuff can be cleaned, in particular washed, when necessary, it is advantageous for the memory device allocated to the measuring module to be detachably connected directly or indirectly to the measuring cuff. However, it is for example hereby also conceivable for the memory device to not be provided on the measuring cuff itself but rather on for example a chest strap or the like. It is also conceivable for the at least one sensor to communicate wirelessly with the memory device in order to be able to transmit the relevant data.

According to one particularly preferential realization of the inventive diagnostic system, it is provided for the evaluation and diagnostic unit to be designed to derive the probability or the risk of the presence of an erectile dysfunction or a biomarker identifying an erectile dysfunction on the basis of the measured variables detected by the at least one sensor. In particular, the evaluation and diagnostic unit is to be designed to derive the probability or the risk of the presence of an erectile dysfunction or a biomarker identifying an erectile dysfunction on the basis of measured variables detected by the at least one sensor over a defined or definable period of time so as to optimize the reliability and reproducibility of the evaluation. Moreover, the pathogenesis can also be traced or charted accordingly.

According to a further development of the latter embodiments, the evaluation and diagnostic unit is in particular designed to assess the risk of vascular disease or the risk of a vascular disease being present using the derived probability or derived risk respectively of the presence of an erectile dysfunction or the derived biomarker identifying an erectile dysfunction.

Advantageously, the evaluation and diagnostic unit is to comprise a display device or similar communication device in order to visually or acoustically output biomarkers derived from the measured variables detected by the at least one sensor. It is particularly befitting in this context for the evaluation and diagnostic unit to be designed as application software, particularly within the realm of mobile operating systems.

The following will reference the accompanying drawings in schematically describing an exemplary embodiment of the diagnostic system according to the invention.

Shown is:

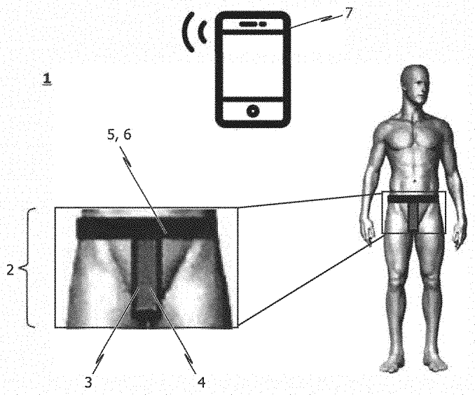

FIG. 1 a schematic view of an exemplary embodiment of the diagnostic system according to the invention.

The diagnostic system 1 according to the invention, as depicted schematically in FIG. 1, is in particular designed as a mobile diagnostic system 1 so as to enable home use.

In the exemplary embodiment of the inventive diagnostic system 1, the measuring module 2 and particularly the measuring cuff 3 comprising the at least one sensor 4 integrated therein are in particular designed to effect a diagnosis of erectile dysfunction.

The inventive diagnostic system 1, as shown schematically in FIG. 1, is in particular designed to provide a biomarker for vascular disease.

Particularly in the case of vascular diseases, the treatment of which can have the patient taking medications for years, along with the associated side effects, a reliable diagnosis of the disease is essential, Biomarkers are becoming more and more important here as they can confirm a difficult diagnosis or even make it possible in the first place. Particularly vascular diseases are often preceded by an early asymptomatic stage of the disease. During this stage, biomarkers help to identify asymptomatic high-risk individuals in a timely and reliable manner.

The inventive diagnostic system 1 enables a corresponding biomarker to be obtained for diagnostic purposes via self-measurement. In particular, the inventive system allows a rapid test such that the result of the biomarker detection is available within a few days, which is important for the attending physician in order to be able to quickly start treatment.

FIG. 1 schematically shows the at least one sensor 4 of the measuring module 2, which is preferably integrated into the measuring cuff 3. Likewise shown schematically is a memory device 5 allocated to the measuring module 2, in which the measured variables detected by the at least one sensor 4 within a predefined or predefinable period of time are buffered.

The memory device 5 allocated to the measuring module 2 is preferably connectible to the evaluation module 7 of the diagnostic system 1 via a wireless communication link 6. A radio link or a Bluetooth connection is for example conceivable in this context. The evaluation module 7 can query and read out the measured variables buffered in the memory device 5 as needed or continuously via said communication link 6.

The evaluation and diagnostic unit, or the evaluation module 7 respectively, is designed to derive the probability or the risk of the presence of an erectile dysfunction or a biomarker identifying an erectile dysfunction on the basis of the measured variables detected by the at least one sensor 4, Said biomarker in turn enables the evaluation and diagnostic unit to assess the risk of vascular disease or the risk of a vascular disease being present, which is shown to the user on a corresponding display device.

1. A mobile diagnostic system (1) for the early detection of vascular diseases comprising a measuring module (2) and an evaluation module (7), wherein the measuring module (2) comprises a measuring cuff (3) made of a textile material and at least one sensor (4) for measuring expansion, pressure, temperature and/or moisture, and wherein the evaluation module (7) comprises an evaluation and diagnostic unit designed to derive a biomarker identifying a vascular disease from the measured variable detected by the at least one sensor (4).

2. The diagnostic system (1) according to claim 1,
wherein the at least one sensor (4) is integrated into the textile material of the measuring cuff (3) or is part of the textile material of the measuring cuff (3).

3. The diagnostic system (1) according to claim 1,
wherein the measuring module (2) comprises a memory device (5) for buffering measured variables detected by the at least one sensor (4) within a predefined or predefinable period of time.

4. The diagnostic system (1) according to claim 3,
wherein the at least one sensor (4) is or is able to be brought into a communication link with the memory device (5) via a data line integrated into the textile material of the measuring cuff (3) or consisting of a textile material, particularly for transmitting the measured variables detected by the at least one sensor (4) as needed or continuously.

5. The diagnostic system (1) according to claim 3,
wherein the memory device (5) comprises an interface (6) via which the measured variables of the at least one sensor buffered in the memory device (5) can be transmitted to the evaluation and diagnostic unit as needed or continuously.

6. The diagnostic system (1) according to claim 3,
wherein the memory device (5) is detachably connected directly or indirectly to the measuring cuff (3).

7. The diagnostic system (1) according to claim 1,
wherein at least one sensor (4) is designed to directly or indirectly detect the occurrence of particularly a nocturnal erection of the user.

8. The diagnostic system (1) according to claim 1,
wherein the evaluation and diagnostic unit is designed to derive the probability or the risk of the presence of an erectile dysfunction or a biomarker identifying an erectile dysfunction on the basis of the measured variables detected by the at least one sensor (4).

9. The diagnostic system (1) according to claim 1,
wherein the evaluation and diagnostic unit is designed to derive the probability or the risk of the presence of an erectile dysfunction or a biomarker identifying an erectile dysfunction on the basis of the measured variables detected by the at least one sensor (4) over a predefined or predefinable period of time.

10. The diagnostic system (1) according to claim 8, wherein the evaluation and diagnostic unit is designed to assess the risk of vascular disease or the risk of a vascular disease being present using the derived probability or derived risk respectively of the presence of an erectile dysfunction or the derived biomarker identifying an erectile dysfunction.

11. The diagnostic system (1) according to claim 1,
wherein the evaluation and diagnostic unit comprises a display device for depicting biomarkers, risk indicators or predictive information derived from the measured variables detected by the at least one sensor (4).

12. The diagnostic system (1) according to claim 1,
wherein the evaluation and diagnostic unit is realized as application software, particularly within the realm of mobile operating systems.

13. The diagnostic system (1) according to claim 4,
wherein the memory device (5) comprises an interface (6) via which the measured variables of the at least one sensor buffered in the memory device (5) can be transmitted to the evaluation and diagnostic unit as needed or continuously.

14. The diagnostic system (1) according to claim 13,
wherein the memory device (5) is detachably connected directly or indirectly to the measuring cuff (3).

15. The diagnostic system (1) according to claim 14,
wherein at least one sensor (4) is designed to directly or indirectly detect the occurrence of particularly a nocturnal erection of the user.

16. The diagnostic system (1) according to claim 15,
wherein the evaluation and diagnostic unit is designed to derive the probability or the risk of the presence of an erectile dysfunction or a biomarker identifying an erectile dysfunction on the basis of the measured variables detected by the at least one sensor (4).

17. The diagnostic system (1) according to claim 16,
wherein the evaluation and diagnostic unit is designed to derive the probability or the risk of the presence of an erectile dysfunction or a biomarker identifying an erectile dysfunction on the basis of the measured variables detected by the at least one sensor (4) over a predefined or predefinable period of time.

18. The diagnostic system (1) according to claim 17,
wherein the evaluation and diagnostic unit is designed to assess the risk of vascular disease or the risk of a vascular disease being present using the derived probability or derived risk respectively of the presence of an erectile dysfunction or the derived biomarker identifying an erectile dysfunction.

19. The diagnostic system (1) according to claim 18,
wherein the evaluation and diagnostic unit comprises a display device for depicting biomarkers, risk indicators or predictive information derived from the measured variables detected by the at least one sensor (4).

20. The diagnostic system (1) according to claim 19,
wherein the evaluation and diagnostic unit is realized as application software, particularly within the realm of mobile operating systems.
